(19) United States
(12) Patent Application Publication
(10) Pub. No.: US 2020/0074623 A1
(43) Pub. Date: Mar. 5, 2020

(54) IMAGE PROCESSING APPARATUS , IMAGE PROCESSING METHOD, AND COMPUTER READABLE RECORDING MEDIUM STORING PROGRAM

(71) Applicant: Konica Minolta, Inc., Tokyo (JP)

(72) Inventor: Hiroaki Matsumoto, Yokohama-shi (JP)

(73) Assignee: Konica Minolta, Inc., Tokyo (JP)

(21) Appl. No.: 16/553,869

(22) Filed: Aug. 28, 2019

(30) Foreign Application Priority Data
Sep. 5, 2018 (JP) 2018-165642

(51) Int. Cl. G06T 7/00; G06T 5/30; G06T 5/00; G06N 3/02; G16H 30/40

(52) U.S. Cl. CPC G06T 7/0012; G06T 5/30; G16H 30/40; G06N 3/02; G06T 5/005

(57) ABSTRACT

An image processing apparatus includes: a hardware processor that: generates a thinned image by decreasing a pixel count on a medical image; inputs the thinned image to a neural network; extracts, using the neural network and from the thinned image, a signal component of a prescribed structure included in the medical image; and executes super-resolution processing on an output image outputted from the neural network to generate a structure image that expresses the signal component. The structure image includes a pixel count identical to the pixel count of the medical image.

### CROSS-REFERENCE TO RELATED APPLICATION

The entire disclosure of Japanese Patent Application No. 2018-165642, filed on Sep. 5, 2018, is incorporated herein by reference.

### BACKGROUND

### Technical Field

The present invention relates to an image processing apparatus, an image processing method, and a computer readable recording medium storing a program.

### Description of the Related Art

It has been proposed to use a neural network for extracting and removing a structure from medical images.

For example, disclosed in Patent Literature 1 (JP 2018-89301A) is to generate an output image by removing a target matter such as a blood vessel from a biological image by using a neural network. Further, disclosed in Patent Literature 2 (JP 4-58943A) is to identify a recognition target by taking a thinned image from which a pixel count is thinned out as an input image of a neural network.

However, when it is desired to extract or remove a large structure with the technique disclosed in Patent Literature 1, the number of pixels in an image and a filter (kernel) used in convolution is increased. Therefore, a calculation amount is increased, and it takes a great time for performing processing.

Further, with the technique disclosed in Patent Literature 2, the thinned image from which the pixel count is thinned out is taken as an input image, so that the processing time can be shortened. However, when a structure is extracted by using a neural network having the thinned image as the input image, the output image (structure image) to be outputted is also the thinned image. Therefore, acquired is only the output image with a large pixel spacing and deteriorated resolution. Further, for example, when a structure in an original medical image is attenuated by subtracting the structure image from the original medical image, it is necessary to perform processing for returning the pixel count to that of the original image by performing enlargement processing on the structure image. However, normally used image enlargement processing such as a linear interpolation method cannot restore edge components, so that the edge components of the structure cannot be removed and therefore the edge components remain when the enlarged structure image is subtracted from the original image.

### SUMMARY

One or more embodiments of the present invention extract a structure from a medical image at a high speed with high precision by using a neural network.

An image processing apparatus according to one or more embodiments of the present invention includes a hardware processor that: generates a thinned image by performing thinning processing for decreasing a pixel count on a medical image; by taking the thinned image as an input image, performs extraction processing of a signal component of a prescribed structure included in the medical image by using a neural network; and performs super-resolution processing on an output image outputted from the neural network to generate a structure image expressing the signal component of the structure in the medical image, the structure image having a pixel count same as (i.e., identical to) the pixel count of the medical image.

An image processing method according to one or more embodiments of the present invention includes: generating a thinned image by performing thinning processing for decreasing a pixel count on a medical image; by taking the thinned image as an input image, performing extraction processing of a signal component of a prescribed structure included in the medical image by using a neural network; and performing super-resolution processing on an output image outputted from the neural network to generate a structure image expressing the signal component of the structure in the medical image, the structure image having a pixel count same as the pixel count of the medical image.

A non-transitory computer readable medium storing a program according to one or more embodiments of the present invention causes a computer to perform (i.e., execute): generating a thinned image by performing thinning processing for decreasing a pixel count on a medical image; by taking the thinned image as an input image, performing extraction processing of a signal component of a prescribed structure included in the medical image by using a neural network; and performing super-resolution processing on an output image outputted from the neural network to generate a structure image expressing the signal component of the structure in the medical image, the structure image having a pixel count same as the pixel count of the medical image.

### BRIEF DESCRIPTION OF THE DRAWINGS

The advantages and features provided by one or more embodiments of the invention will become more fully understood from the detailed description given hereinbelow and the appended drawings which are given by way of illustration only, and thus not are intended as a definition of the limits of the present invention.

FIG. 1 is a block diagram illustrating a functional configuration of an image processing apparatus according to one or more embodiments;

FIG. 2 is a chart illustrating a processing configuration example of a deep learning processor according to one or more embodiments;

FIG. 3 is a flowchart illustrating structure attenuation processing executed by a controller illustrated in FIG. 1;

FIG. 4 is a chart schematically illustrating images outputted by each step of FIG. 3;

FIG. 5 is a chart illustrating a processing configuration example of a deep learning processor according to one or more embodiments;

FIG. 6 is a chart for describing an outline of connection processing according to one or more embodiments; and

FIG. 7 is a chart for describing in detail an example of the connection processing according to one or more embodiments.

### DETAILED DESCRIPTION

Hereinafter, embodiments of the present invention will be described with reference to the drawings. However, the scope of the invention is not limited to the disclosed embodiments.

### [Configuration of Image Processing Apparatus 1]

First, the configuration of one or more embodiments according to the present invention will be described.

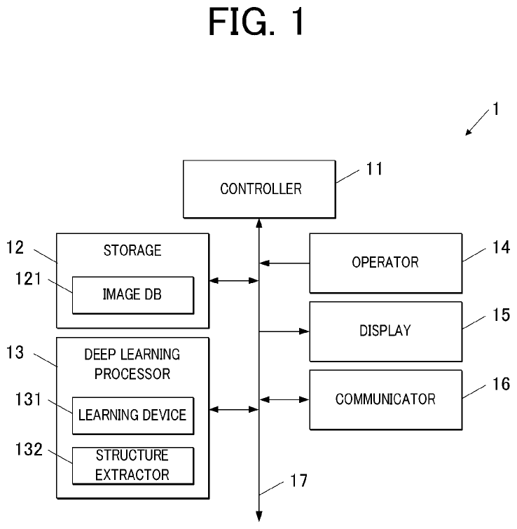

FIG. 1 is a block diagram illustrating a functional configuration of the image processing apparatus 1 according to one or more embodiments. The image processing apparatus 1 is an apparatus performing image processing on medical images. As illustrated in FIG. 1, the image processing apparatus 1 includes a controller 11, a storage 12, a deep learning processor 13, an operator 14, a display 15, a communicator 16, and the like, and each of those components is connected via a bus 17.

The controller 11 is formed with a CPU (Central Processing Unit), a RAM (Random Access Memory), and the like. The CPU of the controller 11 reads out a system program and various kinds of processing programs stored in the storage 12 and expands those programs in the RAM according to operations of the operator 14, and centrally controls actions of each of the components of the image processing apparatus 1 according to the expanded programs. Further, the controller 11 executes various kinds of processing such as structure attenuation processing and the like to be described later on the selected medical image among the medical images stored in an image DB (Data Base) 121 of the storage 12.

The storage 12 is formed with a nonvolatile semiconductor memory, a hard disk, or the like. The storage 12 stores various kinds of programs executed by the controller 11, parameters necessary for the processing executed by the programs, or data of the processing results and the like. The various kinds of programs are stored in a form of readable program codes, and the controller 11 successively executes actions according to the program codes. Further, the storage 12 is provided with the image DB 121 that stores the medical images acquired by radiographing living bodies and medical images in which a prescribed structure is attenuated by associating those with patient information, radiographed body parts, date, and the like.

The deep learning processor 13 includes: a learning device 131 that, by taking a medical image and an image (structure image) expressing signal components of a prescribed structure in the medical image as a set of learning data, extracts the signal components of the prescribed structure from a thinned image (low-resolution image) acquired by performing (i.e., executing) thinning processing for decreasing the pixel count on the inputted medical image by using a large number of learning data sets, and learns parameters of a convolution neural network having a plurality of convolution layers optimized to generate a structure image expressing the signal components (including high-frequency components) of the prescribed structure in the original medical image with the same pixel count as that of the original medical image; and a structure extractor 132 that, by taking the thinned image of the inputted medical image by the convolution neural network using the parameters learned by the learning device 131 as the input image, extracts the signal components of the prescribed structure from the input image, and outputs the structure image expressing the signal components of the prescribed structure in the medical image with the same pixel count (pixel spacing) as that of the medical image. The deep learning processor 13 may be achieved by cooperation work of the CPU of the controller 11 and the programs stored in the storage 12 or may be achieved by a GPU (Graphics Processing Unit).

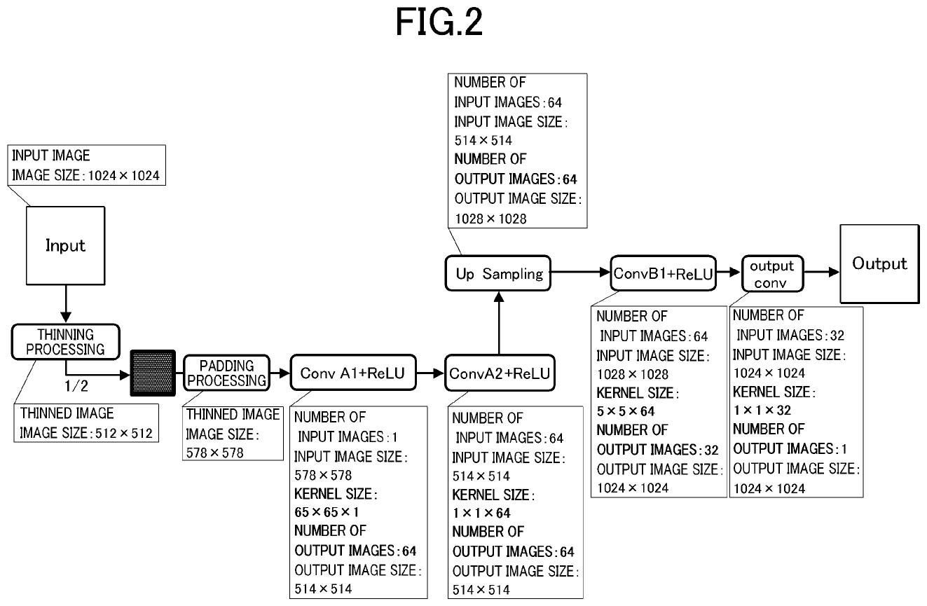

FIG. 2 is a chart illustrating a processing configuration example of the structure extractor 132 according to one or more embodiments. As illustrated in FIG. 2, the structure extractor 132 includes a plurality of convolution layers (a “Conv A1+ReLU” layer, a “Conv A2+ReLU” layer, a “ConyvB1+ReLU” layer, and an “outputconv” layer) of the neural network. In the convolution layers, convolution processing (Convolution) is performed on the input image, and a value acquired by subtracting a bias term from an acquired calculation result is inputted to an activation function (ReLU) to generate and output an output image. “ReLU” is the function that outputs “v” when input “v” is a value of “0” or larger, and output “0” when the input “v” is a negative value. The number of input images of each convolution layer, input image size, kernel size, the number of output images, and output image size are defined in advance (i.e., are predetermined) in design, and the learning device 131 learns a weight coefficient of the kernel and the bias term used for the convolution processing of each convolution layer as the parameters. In the present Description, the image size is expressed by “vertical pixel count×lateral pixel count”, and the kernel size is expressed by “vertical pixel count×lateral pixel count×depth-direction pixel count”. Provided that the image size of the input image is “M×M” and the height width of the kernel size is “N×N”, the output image size can be expressed as “(M−N+1)×(M−N+1).”

Note that the deep learning processor 13 may include not only the convolution layers but also a pooling layer, a fully connected layer, and the like. Also, the activation function used in the convolution layers is not limited to “ReLU” but may also be a sigmoid function, for example. Further, learning of the learning device 131 may be performed by another device in advance, and the deep learning processor 13 may be formed to include only the structure extractor 132.

The operator 14 is formed with a keyboard having a cursor key, numerical input keys, various function keys, and the like and a pointing device such as a mouse, and outputs designation signals inputted by key operations done on the keyboard and mouse operations to the controller 11. Further, the operator 14 may include a touch panel on a display screen of the display 15 and, in such case, outputs the designation signals inputted via the touch panel to the controller 11.

The display 15 is formed with a monitor of an LCD (Liquid Crystal Display) or a CRT (Cathode Ray Tube), and displays input designations from the operator 14, data, and the like according to designation of display signals inputted from the controller 11.

The communicator 16 includes an LAN adaptor, a modem, a TA (Terminal Adapter), and the like, and controls transmission and reception of data with an external device such as a radiography device, not illustrated, connected to a communication network.

### [Actions of Image Processing Apparatus 1]

Next, actions of the image processing apparatus 1 will be described.

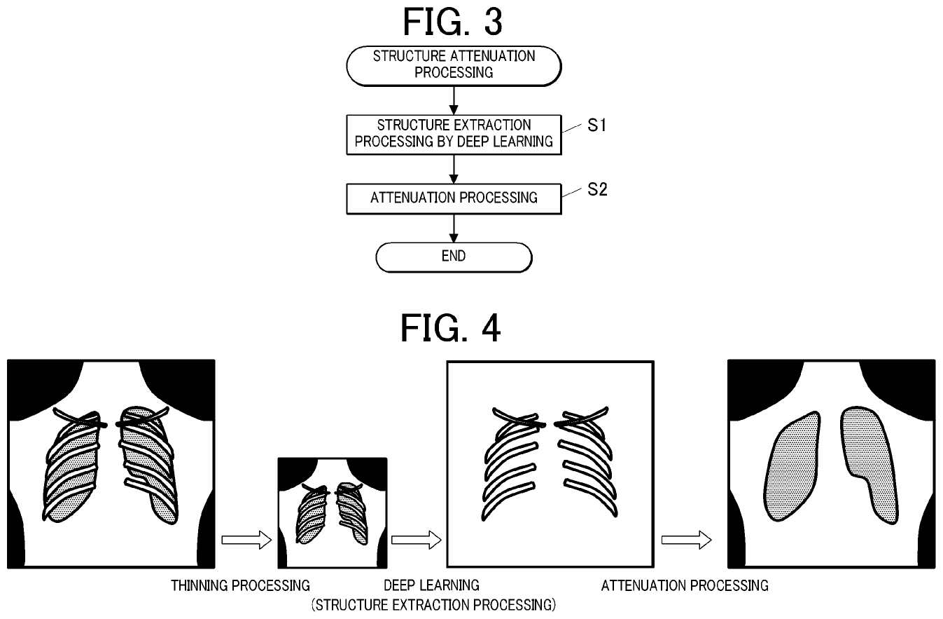

FIG. 3 is a flowchart illustrating the structure attenuation processing executed by the controller 11 when the medical image is selected and attenuation of a prescribed structure is designated by the operator 14. FIG. 4 is a chart schematically illustrating images generated in the structure attenuation processing. The structure attenuation processing is executed by cooperation work of the CPU of the controller 11 and the program stored in the storage 12.

As the medical image, an X-ray image (still image) acquired by taking an X-ray photograph of a living body, an X-ray image (video) acquired by continuously taking X-ray photographs of a living body for a plurality of times, and the like can be applied, for example. Further, examples of the prescribed structure may be a bone such as a costa or a clavicle, an internal organ of a human body such as the heart or a blood vessel, inadvertent of exterior of another radiation detector in a long video acquired by filming by partially superimposing a plurality of radio detectors, and an artificial matter such as an artificial bone, an endoscopic clip, a stent, a pacemaker, or a tube.

While a case where the image size of the medical image as the processing target is “1024×1024” will be described hereinbelow as an example, the size is not limited to that.

First, the controller 11 inputs a selected medical image to the deep learning processor 13 to execute the structure extraction processing (step S1).

Hereinbelow, the structure extraction processing executed by the structure extractor 132 will be described by referring to FIG. 2.

When the medical image selected by the operator 14 is inputted to the deep learning processor 13, the structure extractor 132 executes thinning processing for decreasing the pixel count on the inputted medical image and samples pixel values from the medical image by one-pixel spacing. In the thinned image generated by the thinning processing, the pixel count is decreased than that of the original medical image (herein, ¼ (512×512) of the original medical image). Therefore, the number of times of calculations in each of the convolution layers can be decreased, so that extraction of the structure can be performed at a high speed.

The pixel spacing of sampling in the thinning processing is not limited to be one pixel but may be two pixels, three pixels, and the like. The larger the pixel spacing is, the more the number of times of calculations can be decreased, so that the structure extraction processing can be performed at a higher speed. Further, when performing the thinning processing, a user may designate each of the pixel counts in length and width of the output image. For example, a thinned image may be generated by a reduction image generation method such as nearest neighbor interpolation or a bilinear interpolation by defining the pixel counts in length and width of the original medical image as (Nin, Min), respectively, and the pixel counts of the thinned image as (Nout, Mout) and by designating (Nout, Mout) by the user. Thereby, it is possible to generate the thinned images with a specific pixel spacing at all times without depending on the pixel spacing of the medical images.

Further, preprocessing may be performed on the inputted medical image and the thinned image. As the preprocessing, following processing may be performed, for example.
Normalization processing of entire image or local contrast
Normalization processing of signal values and pixel luminance values
Noise suppression processing by spatial smoothing processing
Noise suppression processing by smoothing processing in time direction in a case of video
Through performing the preprocessing mentioned above, it is possible to perform stable structure extraction without depending on the characteristics of the medical images.

Then, by taking the thinned image as the input image, the structure extractor 132 performs padding processing on the input image. The padding processing is the processing for adjusting the image size of the input image according to the output image size in the “Conv A1+ReLU” layer by setting an area of the pixel value “0” in the periphery of the input image. In one or more embodiments, the thinned image is adjusted to be of “578×578.”

Then, the structure extractor 132 inputs the padding-processed thinned image to the “Conv A1+ReLU” layer. The “Conv A1+ReLU” layer performs convolution processing using sixty-four kinds of “65×65×1” kernels, and outputs sixty-four output images (image size of “514×514”) expressing the features of the signal components of the prescribed structure.

Then, the structure extractor 132 inputs the output images from the “Conv A1+ReLU” layer to the “Conv A2+ReLU” layer as the input images. The “Conv A2+ReLU” layer performs convolution processing using sixty-four kinds of “1×1×64” kernels, and outputs sixty-four output images (image size of “514×514”) expressing the features of the signal components of the prescribed structure.

Then, the structure extractor 132 performs super-resolution processing on the output images from the “Conv A2+ReLU” layer to generate a structure image expressing the signal components (including high-frequency components) of the structure in the original medical image with the same pixel count as that of the original medical image.

First, the structure extractor 132 inputs the output images from the “Conv A2+ReLU” layer to an “Up Sampling” layer. The “Up Sampling” layer performs “Up Sampling” (i.e., upsampling) processing on each of the input images (image size of “514×514”), and outputs the output images (image size of “1028×1028”). As the “Up Sampling” processing, typical enlargement processing can be applied. As a method for interpolating between each of the pixels, it is possible to use zero interpolation, a nearest neighbor method, linear interpolation, a bicubic method, and the like, for example.

Then, the structure extractor 132 inputs the images that are “Up Sampling”-processed in the “Up Sampling” layer to a “Conv B 1+ReLU” layer. The “Conv B1+ReLU” layer performs the convolution processing by using thirty-two kinds of “5×5×64” kernels expressing the features of the signal components of a prescribed structure, and outputs thirty-two output images (image size of “1024×1024”). Further, the structure extractor 132 inputs the output images from the “Conv B1+ReLU” layer to an “outputconv” layer as the input images. The “outputconv” layer performs the convolution processing by using one kind of “1×1×32” kernels expressing the features of the signal components of the prescribed structure, and outputs one structure image (image size of “1024×1024”).

In the “Up Sampling”-processed image, the high-frequency components such as edge components of the prescribed structure in the original medical image are lost (resolution is deteriorated). However, parameters optimized by the learning device 131 are set in the “Conv B 1+ReLU” layer and the “outputconv” layer such that the signal components including the high-frequency components of the prescribed structure in the original medical image are extracted. Therefore, it is possible with the “Conv B1+ReLU” layer and the “outputconv” layer to generate the structure image expressing the signal components (including the high-frequency components) of the prescribed structure in the original medical image from the images outputted from the “Up Sampling” layer.

Provided that the input image size is “m×m”, the number of input images is “C”, the kernel size is “N×N×C”, and the number of output images is “0” in each of the convolution layers, the following can be acquired.

The number of times of multiplication of kernel per pixel: N×N×C

The number of times of addition of single pixel value: N×N×C+1 (1 is addition of bias term)

The calculated pixel count per output image: (M−N+1)×(M−N+1)

Therefore, the calculated number per convolution layer can be acquired as follows.

(N×N×C+N×N×C+1)×(M×N+1)×(M−N+1)×0 (Expression 1)

That is, the calculated number per convolution layer becomes dramatically greater as the kernel size becomes greater and the input image size becomes greater. Further, as the number of convolution layer increases by one, the calculation amount increases by the value calculated with the expression (1).

The deep learning processor 13 of one or more embodiments performs structure extraction by the neural network (convolution layers) with the less pixel count than that of the original medical image, performs the “Up Sampling” processing on the output images of the neural network, and performs the convolution processing on the “Up Sampling”-processed output images by using the parameters optimized (learned) such that the structure image expressing the signal components (including the high-frequency components) of the prescribed structure in the original medical image is outputted, so that it is possible to perform high-speed and high-precision structure extraction processing.

When the structure extraction processing is ended and the structure image is outputted from the deep learning processor 13, the controller 11 performs difference processing for subtracting the structure image from the original medical image by using the outputted structure image to attenuate the signal components of the structure in the medical image (step S2). Specifically, pixel values corresponding to the structure image are subtracted from each of the pixels of the medical image. Then, the structure attenuation processing is ended.

In step S2, the structure is attenuated by subtracting, from the medical image, the structure image of the resolution (including the high-frequency components) same as that of the original medical image and with the same pixel count as that of the original medical image. Therefore, unlike the conventional case, it is possible to prevent the edge components of the structure from remaining in the medical image after the structure is being attenuated.

In a case where the medical image as the processing target is a video, steps S1 to S2 of the above-described structure attenuation processing are performed on each frame image of the video. Therefore, there is a large processing time shortening effect due to reduction of the calculation amount.

After the structure attenuation processing, the controller 11 displays the medical image with the attenuated structure on the display 15 and registers the medical image to the image DB 121 of the storage 12 in response to an operation from the operator 14.

Hereinbelow, one or more embodiments of the present invention will be described.

In one or more embodiments, the learning device 131 of the deep learning processor 13, by taking a medical image and an image (structure image) of a prescribed structure extracted from the medical image as a set of learning data, extracts the signal components of the prescribed structure from a thinned image (low-resolution image) acquired by performing thinning processing for decreasing the pixel count on the inputted medical image by using a great number of learning data sets, and learns parameters of a convolution neural network optimized to output an output image with the pixel count that is “1/n²” of that of the original medical image such that the final number of output images is a square of a natural number “n”.

Further, the structure extractor 132 of the deep learning processor 13 extracts the signal components of the structure by a processing configuration different from that of one or more embodiments by using the parameters learned by the learning device 131.

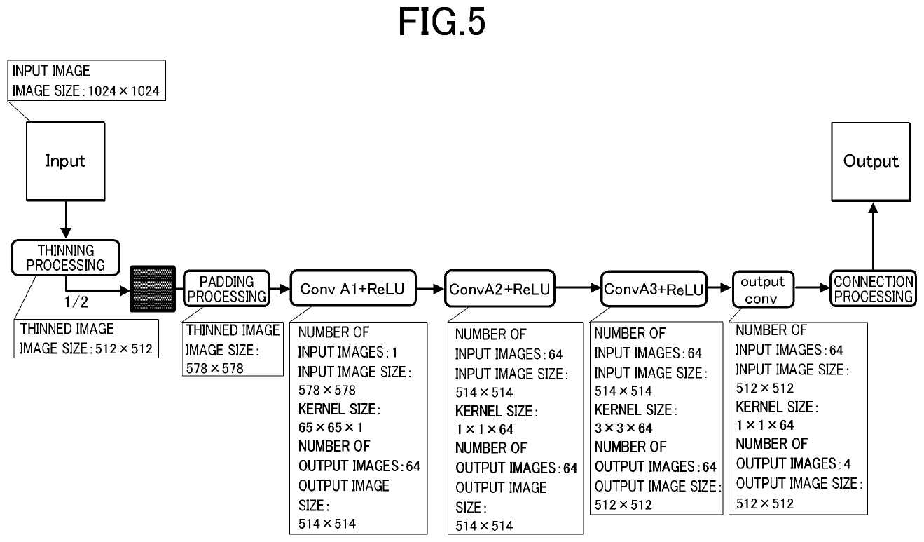

FIG. 5 is a chart illustrating a processing configuration example of the structure extractor 132 according to one or more embodiments. As illustrated in FIG. 5, the structure extractor 132 in one or more embodiments includes a plurality of convolution layers (a “Conv A1+ReLU” layer, a “Conv A2+ReLU” layer, a “Conv A3+ReLU” layer, and an “outputconv” layer) of a neural network. Further, the structure extractor 132 includes a connection processing layer which generates and outputs structure image expressing the signal components (including high-frequency components) of a prescribed structure of an original medical image with the same pixel count as that of the original medical image by performing connection processing on a plurality of (four in this case) output images outputted from the “outputconv” layer.

The “outputconv” layer right before the connection processing is formed such that the number of final output images is a square of a natural number “n” and the pixel count is “1/n²” of that of the original medical image.

Further, the learning device 131 learns in advance (i.e., predetermines) the parameters for acquiring each of n²-pieces of output images such that the signal components of the structure including the high-frequency components of the medical image can be restored by arranging the corresponding pixels of the final (that is, of the “outputconv” layer) n²-pieces of output images of the neural network in an “n×n” array in order defined in advance.

Other configurations of the image processing apparatus 1 according to one or more embodiments described below are the same as that in one or more embodiments described above, so the description of these configurations are not repeated below. Further, the flow of the structural attenuation processing is the same as that illustrated in FIG. 3, and the structural extraction processing executed by structure extractor 132 in step S1 of FIG. 3 of one or more embodiments is different from that of one or more embodiments described below. Therefore, the structure extraction processing according to one or more embodiments will be described hereinbelow by referring to FIG. 5.

When the medical image is inputted to the deep learning processor 13, the structure extractor 132 executes thinning processing for decreasing the pixel count on the inputted medical image and samples the pixel values from the medical image by one pixel spacing. The pixel count of the thinned image generated by the thinning processing is decreased (¼ (512×512) of the original medical image) than that of the original medical image, so that the number of times of calculations in the deep learning processor 13 can be decreased and extraction of the prescribed structure can be performed at a high speed. Detail of the thinning processing is the same as that in one or more embodiments described above, so the description is not repeated below.

Then, by taking the thinned image as the input image, the structure extractor 132 executes padding processing on the input image. In one or more embodiments, the thinned image is adjusted to be of “578×578” by the padding processing.

Then, the structure extractor 132 inputs the padding-processed thinned image to the “Conv A1+ReLU” layer. The “Conv Al+ReLU” layer performs the convolution processing using sixty-four kinds of “65×65×1” kernels, and outputs sixty-four output images (image size of “514×514”) expressing the features of the signal components of the prescribed structure.

Then, the structure extractor 132 inputs the output images from the “Conv A1+ReLU” layer to the “Conv A2+ReLU” layer as the input images. The “Conv A2+ReLU” layer performs the convolution processing using sixty-four kinds of “1×1×64” kernels, and outputs sixty-four output images (image size of “514×514”) expressing the features of the signal components of the prescribed structure.

Then, the structure extractor 132 inputs the output images from the “Conv A2+ReLU” layer to the “Conv A3+ReLU” layer as the input images. The “Conv A3+ReLU” layer performs the convolution processing using sixty-four kinds of “3×3×64” kernels, and outputs sixty-four output images (image size of “512×512”) expressing the features of the signal components of the prescribed structure.

Then, the structure extractor 132 inputs the output images from the “Conv A3+ReLU” layer to the “outputconv” layer as the input images. The “outputconv” layer performs the convolution processing using four kinds of “1×1×64” kernels, and outputs four output images (image size of “512×512”) expressing the features of the signal components of the prescribed structure.

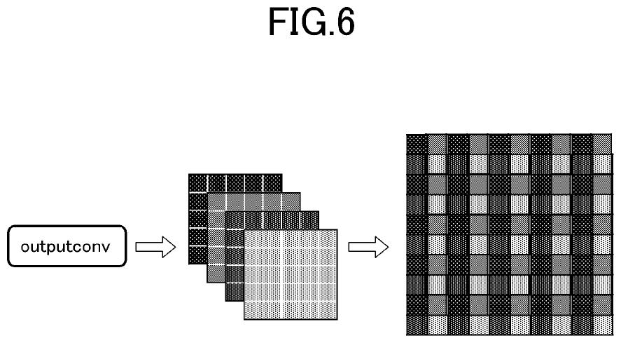

Then, the structure extractor 132 performs connection processing (super-resolution processing) on a plurality of output images outputted from the “Conv A3+ReLU” layer to generate and output a structure image (image size of “1024×1024”) expressing the signal components (including high-frequency components) of the prescribed structure in the original medical image with the same pixel count as that of the original medical image. In the connection processing, as illustrated in FIG. 6, the processing for arranging the corresponding pixels of n²-pieces (herein, n=2) of output images in an “n×n” array in the order defined in advance is performed for each pixel position to execute the super-resolution processing so as to generate the structure image expressing the signal components (including the high-frequency components) of the prescribed structure in the original medical image with the same pixel count as that of the original medical image.

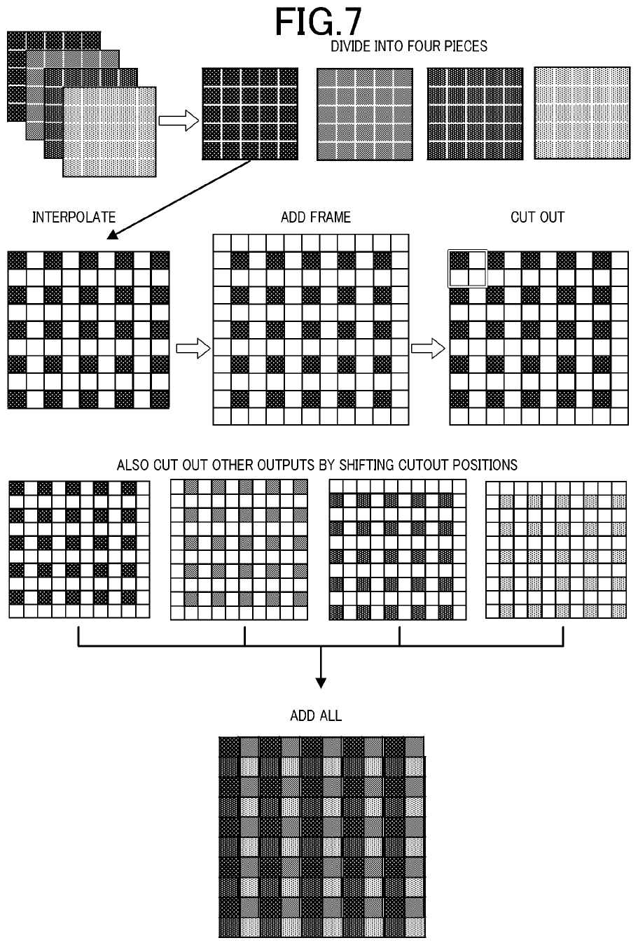

The connection processing may be performed by a method illustrated in FIG. 7, for example. That is, “n−1” pixel is interpolated between neighboring pixels in each of the output images, and a frame of “n−1” pixel is added in the periphery of the interpolated pixels. Then, each of the output images are cut out to the size of the original medical image while shifting the cut position by each of the output images, and a plurality of cutout output images are combined. This makes it possible to generate the signal components (including the high-frequency components) of the prescribed structure in the original medical image with the same pixel count as that of the original medical image. Note that the method of the connection processing is not limited to that.

As described, the deep learning processor 13 of one or more embodiments performs the structure extraction processing with the less pixel count than that of the original medical image and performs the processing for arranging the corresponding pixels of a plurality of output images of a neural network in an array in the order defined in advance for each pixel position to generate the structure image expressing the signal components (including the high-frequency components) of the structure in the original medical image with the same pixel count as that of the original medical image. Therefore, it is possible to perform high-speed and high-precision structure extraction processing. Further, with one or more embodiments, the convolution processing is not performed with the pixel count of the original medical image. Therefore, the calculation amount can be decreased still more compared to that of one or more embodiments described above, thereby making it possible to perform the structure extraction processing at a higher speed. Further, in a case where the medical image as the processing target is a video, steps S1 to S2 of the above-described structure attenuation processing are performed on each frame image of the video. Therefore, there is a large processing time shortening effect due to reduction of the calculation amount.

While one or more embodiments of the present invention are described heretofore, the contents described in one or more embodiments are only examples and the present invention is not limited to those.

For example, in one or more embodiments, numerical examples of the image sizes of the input images and output images, the kernel size, the number of output images, and the like in the processes of the deep learning processor 13 are presented for making it easy to comprehend changes in the pixel count and the calculation amount in the deep learning processor 13. However, those are examples, and the values are not limited to those numerical values.

Further, while one or more embodiments are described by referring to a case of using the structure image generated by the deep learning processor 13 for the structure attenuation processing of the medical image, it is also possible to display the structure image on the display 15 to be used for observation of a prescribed structure.

Further, for example, while a case of using a hard disk, a nonvolatile semiconductor memory or the like as a computer readable medium of the program according to one or more embodiments of the present invention is disclosed in the above description, it is not intended to limit to such case. As another computer readable recording medium, it is possible to apply a portable recording medium such as a CD-ROM. Further, as a medium for providing data of the program according to one or more embodiments of the present invention via a communication line, a carrier wave is also applied.

Also, various changes and modifications are possible in the detail of the configurations and actions of the image processing apparatus without departing from the scope and spirit of the present invention.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

What is claimed is:

1. An image processing apparatus comprising:
a hardware processor that:
generates a thinned image by decreasing a pixel count on a medical image;
inputs the thinned image to a neural network;
extracts, using the neural network and from the thinned image, a signal component of a prescribed structure included in the medical image; and
executes super-resolution processing on an output image from the neural network to generate a structure image that expresses the signal component, wherein the structure image comprises a pixel count identical to the pixel count of the medical image.

2. The image processing apparatus according to claim 1, wherein
the hardware processor further:
executes upsampling processing on the output image; and
executes convolution processing on the output image that has been subjected to the upsampling processing, using a parameter that the neural network learns in advance, to extract the signal component and generate the structure image, wherein
the signal component comprises a high frequency component of the prescribed structure.

3. The image processing apparatus according to claim 1, wherein
the neural network generates a plurality of output images as final output images,
a number of the final output images is a square of a natural number “n,”
a pixel count of the final output images is “1/n²” of the pixel count of the medical image,
the neural network learns in advance a parameter for acquiring each of the final output images,
the parameter enables the signal component to be restored by arranging the pixels of the final output images in an array of “n×n” in a predetermined order, and
the hardware processor arranges the pixels of “n²”-pieces of the final output images in the array of “n×n” in the predetermined order to generate the structure image.

4. The image processing apparatus according to claim 1, wherein the hardware processor further attenuates the signal component based on the structure image.

5. An image processing method comprising:
generating a thinned image by decreasing a pixel count on a medical image;
inputting the thinned image to a neural network;
extracting, by the neural network and from the thinned image, a signal component of a prescribed structure included in the medical image; and
executing super-resolution processing on an output image from the neural network to generate a structure image that expresses the signal component, wherein the structure image comprises a pixel count identical to the pixel count of the medical image.

6. A non-transitory computer readable medium storing a program causing a computer to:
generate a thinned image by decreasing a pixel count on a medical image;
input the thinned image to a neural network;
extract, by the neural network and from the thinned image, a signal component of a prescribed structure included in the medical image; and
execute super-resolution processing on an output image from the neural network to generate a structure image that expresses the signal component, wherein the structure image comprises a pixel count identical to the pixel count of the medical image.
